(19) United States
(12) Patent Application Publication
(10) Pub. No.: US 2009/0246226 A1
(43) Pub. Date: Oct. 1, 2009

(54) Avian vaccines possessing a positive marker gene

(75) Inventors: Motoyuki Esaki, Lenexa, KS (US); Takanori Sato, Fujisawa (JP); Kristi M. Dorsey, Chadds Ford, PA (US)

Correspondence Address: KRATZ, QUINTOS & HANSON, LLP, 1420 K Street, N.W., Suite 400, WASHINGTON, DC, 20005, US

(73) Assignee: ZEON CORPORATION, Tokyo (JP)

(21) Appl. No.: 12/078,307

(22) Filed: Mar. 28, 2008

(51) Int. Cl. A61K 39/245; C12N 7/01; A61P 31/22

(52) U.S. Cl. 424/229.1; 435/235.1

(57) ABSTRACT

The present invention provides a poultry vaccine containing a positive marker gene. More specifically, recombinant turkey herpesvirus modified by the presence of an extraneous antigen gene that may be used as a positive marker to identify and track vaccinated animals is provided. When inoculated into host animals, a poultry vaccine comprising the recombinant turkey herpesvirus provided in the present invention can elicit serological immune responses to the marker gene product that may be detected by serological assays such as enzyme-linked immunosorbent assay and serum agglutination test, thus enabling easy identification and tracking of vaccinated animals.

### CROSS-REFERENCES TO RELATED APPLICATIONS

None

### STATEMENT REGARDING FEDERALLY SPONSORED RESEARCH AND DEVELOPMENT

None

### THE NAMES OF PARTIES TO A JOINT RESEARCH AGREEMENT

Zeon Corporation, Tokyo, Japan; Ceva Sante Animale S.A., France; and Biomune Company, Lenexa, Kansas, USA; are parties to a joint research agreement effective Jun. 15, 2005.

### REFERENCE TO SEQUENCE LISTING

The attached sequence listing, provided in paper copy and on one diskette containing file 080104, is hereby incorporated by reference.

### BACKGROUND OF THE INVENTION

1. Field of the Invention

The present invention relates generally to a poultry vaccine possessing a positive marker that allows easy identification and tracking of vaccinated animals. More specifically, the present invention provides a recombinant turkey herpesvirus (HVT) possessing an extraneous antigen gene that may be used as a positive marker to track vaccination. The recombinant turkey herpesvirus may also contain antigen genes from avian pathogens.

2. Description of the Related Art

In the animal industry, vaccination is a very important and common practice to prevent or reduce symptoms and infection of various diseases. In order to keep flocks or herds of livestock healthy, it is desirable to be able to identify and track vaccinated animals because if all or a part of the animals are not vaccinated properly, there is an increased risk of spreading diseases among the flocks or herds. Occasionally, vaccines may not be properly administered to individual animals due to improper handling of vaccines or inappropriate administration techniques that may involve the vaccination apparatus (Bermudez A. J. and B. Stewart-Brown., 2003, Disease Prevention and Diagnosis. In: Diseases of Poultry, 11th ed. 17-55.). The ability to identify vaccinated animals would help tremendously in examining the cause of a vaccine failure tremendously.

However, identifying and tracking vaccinated animals cannot be easily accomplished. Using conventional vaccines containing either inactivated or modified live whole bacteria or viruses, it is usually impossible to differentiate between antibodies that are produced by vaccination versus those induced by field exposure to a given infectious agent (Bermudez A. J. and B. Stewart-Brown., 2003, Disease Prevention and Diagnosis. In: Diseases of Poultry, 11th ed. 17-55.). Gene-deleted organisms or viruses may be used to differentiate animals infected with wild type field organisms or viruses from vaccinated animals because gene deleted vaccines do not induce antibodies to the deleted antigen protein (L. M. Henderson, 2005, Biologicals, 33: 203-209, EP0887412 and EP1383795). However, it is impossible to identify vaccinated animals after exposure to field organisms or viruses because those animals would develop antibodies to the antigens that were deleted from the gene deleted vaccines. Other vaccines developed using new technologies such as subunit vaccines and DNA mediated vaccines may also be used to differentiate infected from vaccinated animals, but it is also impossible to identify vaccinates after field exposure (L. M. Henderson, 2005, Biologicals, 33: 203-209).

To solve this problem, in the present invention, a poultry vaccine with a positive marker gene is provided. More specifically, the present invention provides a recombinant turkey herpesvirus modified by the presence of an extraneous antigen gene that may be used as a positive marker gene. When inoculated into host animals, a poultry vaccine comprising the recombinant turkey herpesvirus provided in the present invention can elicit serological immune responses to the marker gene product that may be detected by serological assays, thus enabling easy identification and tracking of vaccinated animals. WO 00/53787 describes construction of recombinant porcine reproductive and respiratory syndrome virus expressing an HA epitope of the hemagglutinin gene of influenza A virus and claims that expression of the HA epitope may be used to identify vaccinated animals, but there is no example of inoculating animals with the recombinant virus.

### SUMMARY OF THE INVENTION

The present invention provides a recombinant HVT modified by the presence of a positive marker gene. The recombinant turkey herpesvirus may also contain antigen genes from avian pathogens. The positive marker gene is obtained from organisms or viruses and is capable of eliciting immunity against the protein encoded by the marker gene. The immunity against the protein may be used as a marker to identify and track vaccinated animals easily by conducting simple serological assays. The antigen genes from avian pathogens are able to elicit immunity against the pathogens. A poultry vaccine comprising the recombinant HVT is also provided.

### BRIEF DESCRIPTION OF THE FIGURES

FIG. 1 Baculovirus ELISA titers in chickens vaccinated with the recombinant turkey herpesvirus with the positive marker gene and hemagglutinin gene

FIG. 2 Hemagglutination inhibition titers in chickens vaccinated with the recombinant turkey herpesvirus with the positive marker gene and hemagglutinin gene

### DETAILED DESCRIPTION OF THE INVENTION

### (Positive Marker Genes)

A positive marker gene, which is inserted into a HVT DNA genome and allows easy identification and tracking of vaccinated animals, encodes a protein that is capable of eliciting humoral immune responses that may be detected by serological assays. The positive marker gene is obtained from any organisms or viruses. Preferably, the positive marker gene is obtained from viruses, bacteria, fungi, or protozoa. More preferably, the positive marker gene is obtained from viruses. Most preferably, the positive marker gene is obtained from Autographa californica nuclear polyhedrosis virus.

The positive marker gene may be any gene or any portion of a gene in organisms or viruses as long as the encoded protein elicits humoral immune responses. Typically, highly immunogenic proteins are located on the surface of organisms or viruses. In one embodiment, the gp64 gene obtained from Autographa californica nuclear polyhedrosis virus is used as the positive marker gene. In another embodiment, the pp34 gene obtained from Autographa californica nuclear polyhedrosis virus is used as the positive marker gene.

(Antigen Genes from Avian Pathogens)

In the present invention, antigen genes from avian pathogens may also be inserted into a HVT DNA genome. Antigen genes encode proteins that are capable of eliciting immune responses in animals. The antigen genes may be obtained from any organisms that cause diseases in poultry. These avian pathogens include, but are not limited to, infectious bursal disease virus, Newcastle disease virus, avian influenza virus, laryngotracheitis virus, infectious bronchitis virus, chicken anemia virus, Mycoplasma gallisepticum, Mycoplasma synoviae, and Eimeria species. A number of antigen genes into a HVT DNA is not limited.

In one embodiment, the hemagglutinin (HA) gene obtained from avian influenza virus is inserted into a HVT DNA genome. Preferably, the HA gene is obtained from an avian influenza virus of the H5 subtype. More preferably, the HA gene is obtained from the avian influenza virus A/Turkey/Wisconsin/68 (H5N9) strain.

### (Turkey Herpesvirus)

As long as it is non-pathogenic to chickens, any HVT can be used in the present invention. For instance, the following HVT strains, FC126, PB-THV1, H-2, YT-7, WTHV-1, and HPRS-26, are suitable for the backbone virus. Among these, the FC126 strain is favorable for use in the present invention.

### (Region for Gene Insertion)

In the present invention, a positive marker gene and antigen genes from avian pathogens are inserted into a HVT DNA genome. Preferably, the genes are inserted into a region in the HVT genome that is non-essential for virus growth. Several non-essential regions in the HVT genome have been reported. For instance, the genes can be inserted into, but not limited to UL43 (WO 89/01040), US2 (WO 93/25665) or inter-ORF region between UL44 and UL46 (WO 99/18215). Most preferably, the genes can be inserted into the inter-ORF region between UL45 and UL46.

For the present invention, a non-essential region may be newly identified by the following general procedure. First, HVT DNA fragments of appropriate lengths are cloned into an E. coli plasmid and physically mapped by restriction enzyme analysis. Then, a gene cassette consisting of a promoter and a reporter gene is inserted into an appropriate restriction site of the cloned DNA fragment resulting in a homology plasmid. If homologous recombination with the obtained homology plasmid results in a recombinant virus expressing the inserted reporter gene and if the virus is stable in vitro and in vivo, the originally selected DNA fragment should be a non-essential region suitable for gene insertion.

### (Promoter)

Expression of the positive marker gene and the antigen genes from avian pathogens inserted in a HVT genome is under control of promoters located upstream of those genes. The promoters may be any endogenous promoters or any exogenous promoters as long as recombinant HVT expresses enough proteins to elicit immune responses in host animals. Preferably, the promoters are selected from the chicken beta-actin promoter (T. A. Kost et al., 1983, Nucleic Acids Res. 11:8287-8301), the core sequence of beta-actin promoter (U.S. Pat. No. 6,866,852), a modified chicken beta-actin promoter (U.S. Pat. No. 6,866,852), or the cytomegalovirus immediate early promoter (CMV promoter) (M. Boshart et al., 1985, Cell 41: 521-530).

(Construction of rHVT)

For the present invention, any known method of generating recombinant HVT is applicable. A typical example is as follows. (1) First, as described above, a recombinant plasmid that contains a non-essential region of the HVT genome is constructed. Then, preferably with a promoter at the 5′ terminus and a polyadenylation signal at the 3′ terminus, a positive marker gene and antigen genes from avian pathogens are inserted into the non-essential region to generate a homology plasmid. (2) The homology plasmid is transfected into chicken embryo fibroblast (CEF) cells infected with parent HVT or co-transfected into CEF cells with infectious HVT genomic DNA. Transfection can be performed by any known method. (3) The transfected CEF cells are planted on tissue culture plates and incubated until virus plaques become visible. (4) The identifiable plaques include recombinant virus as well as parent wild-type virus. The recombinant virus may be purified from wild type virus by any known method to detect expression of inserted foreign genes.

### (Positive Marker Vaccine)

The vaccine consisting mainly of the recombinant HVT containing a positive marker gene in the present invention allows easy identification and tracking of vaccinated host animals by conducting simple serological assays using sera collected from the host animals. The vaccine may include chicken cells and/or ingredients of culture media. As long as not pharmacologically detrimental, the vaccine may contain any ingredients such as preservatives. In addition, the vaccine of the present invention can be used in a mixture with any recombinant or non-recombinant viruses such as the MDV serotype 1 or serotype 2 vaccine strains.

Any known method is applicable to the preparation of the recombinant vaccine in the present invention. For instance, the recombinant HVT may be inoculated into permissive culture cells such as CEF cells and grown to an appropriate titer. Then, the cells are removed from tissue culture plates or roller bottles with cell scrapers or by trypsin treatment and collected by centrifugation. The pelleted cells are then suspended in culture medium containing dimethyl sulfoxide, frozen slowly, and then stored in liquid nitrogen. Alternatively, the recombinant HVT may be released from the infected cells by disrupting the cells in diluents containing stabilizers such as sucrose and bovine albumin. This released HVT is called cell-free HVT. Cell-free HVT may be lyophilized and stored at 4° C.

The recombinant HVT vaccine can be administered to chickens by any known method of inoculating Marek's disease vaccine. For instance, the vaccine of the present invention is diluted to give 10¹-10⁵, or more favorably 10²-10⁴ plaque forming units (pfu) per dose, and inoculated subcutaneously behind the neck of one-day-old chicks or into embryonating eggs via the in ovo route with syringes or with any apparatus for injection.

Sera may be collected from vaccinated animals at any time after 4 weeks of vaccination. Preferably, sera are collected between 4 weeks and 7 weeks post vaccination. Humoral antibodies in those sera against a protein encoded by a positive marker gene may be detected by any serological assays that perceive antibody-antigen interaction including enzyme-linked immunosorbent assay (ELISA), agar-gel immunodiffusion test, serum agglutination test, hemagglutination test, and radioimmunoassay. Preferably, ELISA is used for detecting the humoral immune responses.

Any antigens may be used in the serological assays as long as the antigens are capable of detecting humoral antibodies against a protein encoded by a positive marker gene in host animals. Preferably, antigens are selected from the group that includes a full-length or a truncated form of the protein encoded by a positive marker gene and organisms or viruses that the positive marker gene is obtained from. In one embodiment, Autographa californica nuclear polyhedrosis virus particles are used in ELISA to detect immune responses in chickens vaccinated with rHVT containing the gp64 gene as the positive marker gene.

### Description of the Preferred Embodiments

Gene cloning and plasmid construction was essentially performed by the standard molecular biology techniques (Molecular Cloning: A Laboratory Manual. 3rd Edition, Cold Spring Harbor Laboratory Press, Woodbury, N.Y. 2001). The turkey herpesvirus FC126 strain (R. L. Witter et al., 1970, Am. J. Vet. Res. 31, 525-538) was used as a backbone virus to generate a recombinant turkey herpesvirus.

### EXAMPLE 1

### Cloning of gp64 Gene from Baculovirus (AcNPV)

The DNA of Baculovirus (Autographa californica nuclear polyhedrosis virus; AcNPV) was commercially available (Orbigen, Inc., Cat. #: BVD-10001). To obtain gp64 gene fragments from AcNPV, polymerase chain reaction (PCR) was conducted using Ex Taq Polymerase (TAKARA BIO INC., Shiga 520-2193, Japan, Cat#: RR001A) and with two kinds of PCR primer sets. One pair was GP64-F1 (SEQ ID NO. 3) and GP64-R2 (SEQ ID NO. 4), and amplified 857-basepair (bp) DNA fragment. The other pair was GP64-F2 (SEQ ID NO. 5) and GP64-R1 (SEQ ID NO. 6), and amplified 724-bp DNA fragment. To connect the resulting two DNA fragments, the reaction mixtures were mixed as a new template, and the next CR was performed using the primer set of GP64-F1 (SEQ ID NO. 3) and GP64-R1 (SEQ ID NO. 6). The amplified 1.55 kilobase (kb) gp64 DNA was inserted into pCR2.1-TOPO vector (Invitrogen, Cat.#: K4500-01), resulting in pCR2.1-GP64. Nucleotide sequences of the gp64 genes in four candidate clones of the plasmid pCR2.1-GP64—were determined using Beckman Sequencer CEQ2000 (Beckman) with six primers; M13 Forward primer (SEQ ID NO. 7), M13 Reverse primer (SEQ ID NO. 8), GP64-F3 (SEQ ID NO. 9), GP64-R3 (SEQ ID NO. 10), GP64-F2 primer (SEQ ID NO. 5), and GP64-R2 primer (SEQ ID NO. 4).

The sequences in two of four clones of the plasmid pCR2.1-GP64 were identical to that of gp64 gene registered in GeneBank (Acc.#: NC_{—}001623). The nucleotide sequence and the deduced amino acid sequence of the cloned gp64 gene are shown in SEQ ID NO. 1 and NO. 2.

### EXAMPLE 2

### Cloning of pp34 Gene from Baculovirus (AcNPV)

To obtain pp34 gene fragments from AcNPV, PCR was conducted using Pfu DNA Polymerase (Stratagene, Cat.#: 600153) and with a PCR primer set of pp34-F primer (SEQ ID NO. 11) and pp34-R primer (SEQ ID NO. 12). The amplified 784-bp pp34 DNA was inserted into pPCR-Script Amp vector (Stratagene, Cat.#: 211188), resulting in pPCR-pp34. Nucleotide sequences of the pp34 genes in two candidate clones of the plasmid pPCR-pp34 were determined using Beckman Sequencer CEQ2000 (Beckman) with four primers; M13 Forward primer (SEQ ID NO. 7), M13 Reverse primer (SEQ ID NO. 8), pp34-F (SEQ ID NO. 11), and pp34-R (SEQ ID NO. 12). The sequences in both of two clones were identical to that of pp34 gene registered in GeneBank (Acc.#: NC-001623). The nucleotide sequence and the deduced amino acid sequence of the cloned pp34 gene are shown in SEQ ID NO. 13 and NO.14

### EXAMPLE 3

### Construction of Homology Vectors

3-1. Construction of an Intermediate Plasmid p46Sfi

HVT-DNA was prepared as described in Example 1 of U.S. Pat. No. 6,632,664. A new SfiI restriction enzyme site into which foreign genes were inserted, was generated by PCR in vitro mutagenesis using two primer pairs. The primer pairs were HVT45Sph (SEQ ID NO. 15) and 45SfiR (SEQ ID NO.16), and 46SfiF (SEQ ID NO. 17) and HVT46Xho (SEQ ID NO. 18). Two PCR reactions were conducted separately using each pair of primers and HVT-DNA as a template. Then two PCR products (0.4 kb and 0.6 kb, respectively) were mixed and used as a template for the secondary PCR with a primer pair of HVT45Sph (SEQ ID NO. 15) and HVT46Xho (SEQ ID NO. 18), yielding the 0.98 kb fragment. The amplified fragment was digested with Sphl and Xhol and inserted into Sphl and Xhol digested pNZ45/46Sfi (Example 2 of U.S. Pat. No. 6,632,664), resulting in p46Sfi.

3-2. Construction of p46Bac

Two synthetic oligonucleotides, Sfi-polA-Sal-Pst-Sfi-U (SEQ ID NO. 19) and Sfi-polA-Sal-Pst-Sfi-L (SEQ ID NO. 20) were designed to introduce polA signal and two restriction enzyme sites into Sfil site of p46Sfi. They were mixed and annealed to generate a Sfi-polA-Sal-Pst-Sfi adapter. Sfil-cut p46Sfi was dephosphorylated and sequentially ligated with the adapter, resulting in p46Sfi-PA. The plasmid pNZ45/46BacpA (U.S. Pat. No. 7,153,511) was digested with Pstl and Sall, and the chicken beta-actin promoter (Bac) fragment of 1.57 kb was recovered from 1.2% agarose gel. The plasmid p46Sfi-pA was also digested with Pstl and Sall, and ligated with the recovered Bac promoter fragment, resulting in p46Bac.

3-3. Construction of Homology Vector p46BacGP64

PCR primers, GP64-Xba-F primer (SEQ ID NO. 21) and GP64-Sal-R primer (SEQ ID NO. 22), contain a sequence for a restriction enzyme, Xbal and SalI at their 5′ ends, and can anneal to the sequence near start and stop codon of the gp64 gene, respectively. Using these primers and pCR2.1-GP64 as a template, PCR was conducted to introduce these restriction enzyme sites before stop codon and after termination codon of gp64 gene. The amplified DNA was purified and digested with Xbal and Sall. The digested DNA fragment was ligated with p46Bac digested with Xbal and Sall, resulting in p46BacGP64.

3-4. Construction of Homology Vector p46CoaGP64

The Core sequence (about 300 bp) of beta-actin promoter was prepared by digesting pGICOA described in Example 1 of U.S. Pat. No. 6,866,852 with Pstl and Xbal, and ligated with two fragments digested both with Pstl and HindIII, and with HindIII and XbaI of p46BacGP64, resulting in p46CoaGP64.

3-5. Cloning of HA Gene Derived from the Avian Influenza Virus

The avian influenza virus A/Turkey/Wisconsin/68 (H5N9) strain was propagated in the allantoic sac of specific pathogen free embryonating chicken eggs. Total genomic RNA from the A/Turkey/Wisconsin/68 virus was extracted using RNeasy Mini Kit (QIAGEN, Cat#74104). First-strand cDNA was synthesized with SuperScript First-Strand System for RT-PCR (Invitrogen, Cat#11904-018). Using the resulting cDNA as a template, the HA gene was amplified by polymerase chain reaction (PCR) with PfuUltra High-Fidelity DNA Polymerase (Stratagene, Cat#600380) and PCR primers. These PCR primers, BamHA-F primer (SEQ ID NO. 23) and SalHA-R primer (SEQ ID NO. 24), anneal to the start and stop sequences of the HA gene and each primer contains a sequence at the 5′ ends for a restriction enzyme, BamHI or SalI, respectively. After the PCR reaction, Taq polymerase (Promega, Cat# M2665) was added to the PCR mixture to add 3′ A-overhangs to the PCR products.

The amplified 1.8 kb HA cDNA was inserted into pCR2.1-TOPO vector (Invitrogen, Cat# K4500-01), resulting in pCR2.1-H5Wis68. Nucleotide sequences of the HA genes in a few clones of the plasmid pCR2.1-H5Wis68 and the PCR product were determined using ABI PRISM 3730XL DNA Analyzer (Applied Biosystems) with six primers; BamHA-F primer (SEQ ID NO. 23), SalHA-R primer (SEQ ID NO. 24), M13 Forward primer (SEQ ID NO. 7), M13 Reverse primer (SEQ ID NO. 8), HA-F primer (SEQ ID NO. 25), and HA-R primer (SEQ ID NO. 26).

The sequences in the clones of the plasmid pCR2.1-H5Wis68 were identical to each other and to the sequence of the PCR product. Although the deduced amino acid sequence was different from the reported sequence of A/Turkey/Wisconsin/68 (H5N9) (M. Garcia et al., 1997, Virus Res. 51: 115-124, GenBank Accession# U79456) by four amino acids, the amino acids we obtained were the same as the amino acids of a majority of H5 subtype HA proteins. The nucleotide sequence and the deduced amino acid sequence of the HA gene obtained from A/Turkey/Wisconsin/68 (H5N9) are shown in SEQ ID NO. 27 and NO. 28.

3-6. Construction of Homology Vector p46CMVH5Wis68

The cytomegalovirus immediate early promoter (CMV promoter) was obtained from pBK-CMV (Stratagene, Cat. #212209). Three BglI restriction enzyme sites in the CMV promoter were disrupted for ease of the plasmid construction process by PCR in vitro mutagenesis using four pairs of primers. The primer pairs were PrCMV1 (SEQ NO.29) and PrCMV3 (SEQ NO.30), PrCMV4 (SEQ NO.31) and PrCMV5 (SEQ NO.32), PrCMV6 (SEQ NO.33) and PrCMV2′ (SEQ NO.34), and PrCMVo1 (SEQ NO.35) and PrCMVR1 (SEQ NO.36). Four PCR reactions were conducted separately using each pair of primers and pBK-CMV as a template. Then four PCR products were combined and used as a template for the secondary PCR with primers PrCMV1 and PrCMVR1, yielding the 604 bp fragment with a modified CMV promoter sequence. The nucleotide sequence of the CMV promoter used to express HA gene is provided in SEQ ID. NO. 37.

The CMV promoter fragment was digested with PstI and XbaI and inserted into PstI and XbaI digested pUC18polyASfi (U.S. Pat. No. 6,866,852), resulting in pGICMV(−). The SV40 polyA signal was obtained from pBK-CMV by PCR using primers PolyA-SalKpn (SEQ NO.38) and PolyA-SfiF2 (SEQ NO.39). The PCR fragment containing SV40 polyA signal was digested with SalI and SfiI and ligated to pGICMV(−) digested with SalI and SfiI resulting in pGICMVpA.

To modify the terminal sequence of CMV promoter-polA fragment, PCR was conducted using pGICMVpA as a template, and a primer pair of PrCMV1Bgl (SEQ NO. 40) and PolA-SfiR (SEQ NO. 41). The amplified PCR fragment of 955 bp was digested with Bgll and inserted into the Sfil site of p46Bac, resulting in p46CMV.

Then, the HA gene from A/Turkey/Wisconsin/68 (H5N9) was excised from pCR2.1-H5Wis68 using Sall and BamHl. The 1701 bp HA gene was inserted into p46CMV digested with Sall and BamHI, resulting in p46CMVH5Wis68.

3-7. Construction of Homology Vector p46BacGP64CMVH5Wis68

Then, p46CMVH5Wis68 was digested with BglI and the “CMV promoter-HA gene-polA signal” fragment of 2.6 kb was inserted into the Sfil site of p46BacGP64, resulting in p46BacGP64CMVH5Wis68. This plasmid was used as a homology vector to generate recombinant turkey herpesvirus.

3-8. Construction of Homology Vector p46CoaGP64CMVH5Wis68

Again, p46CMVH5Wis68 was digested with BglI and the “CMV promoter-HA gene-polA signal” fragment of 2.6 kb was inserted into the Sfil site of p46CoaGP64, resulting in p46CoaGP64CMVH5Wis68. This plasmid was also used as a homology vector to generate recombinant turkey herpesvirus.

### EXAMPLE 4

### Generation and Isolation of Recombinant Turkey Herpesvirus

Viral DNA of the HVT FC126 strain was prepared as described by Morgan et al. (Avian Diseases, 1990, 34:345-351).

10⁷ secondary chicken embryo fibroblast (CEF) cells were suspended in Saline G (0.14 M NaCl, 0.5 mM KCl, 1.1 mM Na₂HPO₄, 1.5 mM NaH₂PO₄, 0.5 mM MgCl₂, and 0.011% glucose) and co-transfected with HVT viral DNA and 5 to 25 μg of the homology vector, p46BacGP64CMVH5Wis68 or p46CoaGP64CMVH5Wis68 by electroporation. Electroporation was performed using Bio-Rad Gene Pulser. Transfected cells were incubated for 10 minutes at room temperature and transferred to wells of 96-well plates. After incubating at 37° C. for 7 days in 4-5% CO₂, or until the plaques became visible, the cells were detached from the plates by trypsinization, transferred equally to two 96-well plates with secondary CEF and incubated for 3 to 4 days until plaques were observed. Screening was conducted by the black plaque assay, staining only plaques expressing HA protein. Briefly, one of the two plates was fixed with methanol:acetone mixture (1:2) and incubated with chicken anti-HA antiserum or mouse anti-GP64 monoclonal antibody (AcV1; eBioscience, Cat#14-6991-82). Next, incubated with biotinylated anti-chicken IgG antibody (Vector Laboratories, Cat# BA-9010) or biotinylated anti-mouse IgG antibody (Vector Laboratories, Cat# BA-9200) and then with VECTASTAIN ABC-AP kit (Vector Laboratories, Cat# AK-5000), plaques expressing HA protein were stained by addition of BCIP/NBT solution (Bio-Rad Laboratories, Cat#170-6539 and 170-6532). Wells containing stained recombinant plaques were identified and cells from the corresponding wells on the other 96-well plate were trypsinized. The cells were then diluted in fresh secondary CEF cells and transferred to 96-well plates to complete the first round of purification.

The purification procedure was repeated until all plaques were stained positively in the black plaque assay. Purified recombinant viruses were designated as rHVT/BacGP64CMVH5Wis68 and rHVT/CoaGP64CMVH5Wis68, respectively.

### EXAMPLE 5

### Verification of Genome Structure of Recombinant HVT by Southern Blot Analysis

Chicken embryo fibroblast cells in a 100-mm dish that were infected with the recombinant viruses, rHVT/BacGP64CMVH5Wis68 and rHVT/CoaGP64CMVH5Wis68, or the HVT FC126 parent strain were used in the Southern blot analysis to confirm the insertion of the HA gene and the gp64 gene in the desired insertion site. The cells were collected by a cell scraper and by centrifugation at 913×g for 5 minutes. The harvested cells were washed with phosphate buffered saline (PBS) and resuspended in 1.0 milliliter (ml) of lysis buffer (0.5% TritonX-100, 100 mM 2-mercaptethanol, and 20 mM EDTA in PBS). The cell suspension was vortexed for a total of 30 seconds and incubated for 15 minutes at room temperature. Cell nucleus and cell debris were removed by centrifuging at 2,060×g for 5 minutes and the supernatant was transferred to a 1.5-ml tube. Viruses were collected by centrifugation at 20,800×g for 20 minutes at 4° C. The pellet was suspended in 0.33 ml of a nuclease solution (12.5 mM Tris-Cl (pH7.5), 1 μg/ml DNase I and 1 μg/ml RNase A) and incubated at 37° C. for 30 minutes. Then, 83 μl of SDS-protease solution (50 mM EDTA, 5% SDS, 0.5 mg/ml protease K, and 25 mM 2-mercaptoethanol) was added to the virus suspension and incubated at 55° C. for 30 minutes to disrupt virus envelopes. Phenol chloroform extraction was conducted twice and DNA was precipitated by adding 2.5 volume of cold 100% ethanol and NaCl at a final concentration of 0.16 M. After centrifuging at 20,800×g for 30 minutes at 4° C., the pellet was washed with 70% ethanol and air-dried. The pellet was dissolved in TE buffer (10 mM Tris-Cl (pH8.0), and 1 mM EDTA).

The viral DNA in TE buffer and the homology plasmid (positive control) were digested with EcoRI and PvuI, and separated by agarose gel electrophoresis using 0.6% agarose gel. DNA fragments on the gel were transferred to a Biodyne A nylon membrane (Pall, Cat# BNXF3R). The membrane was hybridized with either Digoxigenin (DIG)-labeled HA probe, GP64 probe or IS45/46 probe. The DIG-labeled HA probe, the GP64 probe and the IS45/46 probe were prepared with PCR DIG Probe Synthesis Kit (Roche Applied Science, Cat#11636090910) using primers HA2-P-F (SEQ ID NO. 42) and HA2-P-R (SEQ ID NO. 43), primers GP64-F (SEQ ID NO. 44) and GP64-R (SEQ ID NO. 45), and primers 45/46-F (SEQ ID NO. 46) and 45/46-R (SEQ ID NO. 47), respectively.

The membrane was washed with 2×SSC solution at room temperature and then with 0.5×SSC solution at 68° C. The membrane was blocked and incubated with anti-Digoxigenin-AP, Fab fragments (Roche Applied Science, Cat#11093274910) for 30 minutes at room temperature. After washing two times with maleic acid washing buffer (0.1 M maleic acid, 0.15 M NaCl, and 0.3% Tween20, pH 7.5), DNA bands that were hybridized with the probes were visualized by incubating the membrane with BCIP/NBT solution. The HA probe and the GP64 probe hybridized with a 5.5 kb band in rHVT/BacGP64CMVH5Wis68 and a 4.2 kb band in rHVT/CoaGP64CMVH5Wis68, while no bands were detected with the HVT parent. The IS45/46 probe hybridized with 5.5 kb and 1.2 kb bands in rHVT/BacGP64CMVH5Wis68 and 5.5 kb and 1.2 kb bands in rHVT/CoaGP64CMVH5Wis68, and with 1.0 kb band in the HVT parent. These results demonstrated that rHVT/BacGP64CMVH5Wis68 and rHVT/CoaGP64CMVH5Wis68 obtained in EXAMPLE 4 had expected genomic structures.

### EXAMPLE 6

### Protein Expression by Recombinant HVT

Expression of the HA protein and GP64 protein by the recombinant viruses, rHVT/BacGP64CMVH5Wis68 and rHVT/CoaGP64CMVH5Wis68, was confirmed by the black plaque assay and the Western blot assay. Procedures for the black plaque assay are described in EXAMPLE 4. The western blot was conducted using CEF cells infected with the recombinant viruses and chicken anti-HA antiserum or mouse anti-GP64 monoclonal antibody (AcV5; eBioscience, Cat#14-6995-82). Briefly, CEF cells in 100-mm dishes were infected with one of the recombinant viruses or the parent HVT FC126 strain at a multiplicity of infection of approximately 0.01. Two to three days post inoculation, cells were harvested with cell scrapers and centrifuged at 913×g for 5 minutes. The pellet was washed with PBS twice and resuspended with 50 to 100 μl of PBS. After adding the same volume of 2×SDS sample buffer (130 mM Tris-Cl (pH6.8), 6% SDS, 20% Glycerol, 10% 2-Mercaptoethanol and 0.01% Bromo Phenol Blue), cell suspension was boiled for 5 minutes. The samples were separated by SDS-PAGE using 8% polyacrylamide gel and transferred to a PVDF membrane (Immobilon-P, Millipore). The membrane was dried completely and then incubated with chicken anti-HA antiserum or mouse anti-GP64 monoclonal antibody AcV5. After the anti-HA antiserum or mouse anti-GP64 monoclonal antibody AcV5 was washed off, the membrane was incubated with alkaline phosphatase-conjugated anti-chicken IgG Fc antibody (Bethyl, Cat# A30-104AP) or alkaline phosphatase-conjugated anti-mouse IgG antibody (Bethyl, Cat# A90-116AP). Protein bound with chicken anti-HA antiserum mouse anti-GP64 monoclonal antibody AcV5 was visualized by adding BCIP/NBT solution. The HA protein with the size of 74 kilodaltons (kDa) or the GP64 protein with the size of 64 kDa was observed only in the lane with the recombinant virus infected cells.

### EXAMPLE 7

### Development of ELISA System

Serological responses to the GP64 protein expressed by the recombinant viruses, rHVT/BacGP64CMVH5Wis68 and rHVT/CoaGP64CMVH5Wis68, in vaccinated chickens were examined by the ELISA assay. The ELISA assay was conducted on serum collected from rHVT/BacGP64CMVH5Wis68 and rHVT/CoaGP64CMVH5Wis68 vaccinated chickens. Briefly, the ELISA plates were coated with wild type Baculovirus (BD Biosciences, Cat#554744) which was grown in sf9 insect cells (Invitrogen, Cat#12659-017) and purified by ultracentrifugation. The ELISA plate was next fixed with 150 μl of methanol and then placed at −20° C. until ELISA was conducted. Plates were removed from −20° C. and allowed to come to room temperature. The ELISA plate was then blocked with 190 μl of blocking buffer [2% Nonfat dry milk, 0.5% Ovalbumin (Albumin, Chicken Egg Grade V) in PBS] at 4° C. overnight followed by two washes with washing solution (PBS-0.05% Tween 20). Serum samples were diluted 1:128 in dilution buffer [2% Nonfat dry milk, 0.5% Ovalbumin (Albumin, Chicken Egg Grade V), 0.1% Albumin, Bovine (Initial fraction by heat shock) and 0.05% Tween-20 in PBS] and 50 μl of the diluted serum was added to the ELISA plate. The plate was sealed and incubated at 37° C. for one hour. The serum was removed and the ELISA plate was washed five times with washing solution. Then 50 μl of 1:5000 diluted goat anti-chicken IgG Fc HRPO conjugated (Bethyl, Cat # A30-104P) was added to all wells. The plate was sealed and incubated at 37° C. for one hour. The antibody was removed and the ELISA plate was washed five times with washing solution. Then 50 μl of room temperature 1-Step Turbo TMB (Pierce, Cat#34022) was added to all wells. The plate was sealed and incubated at room temperature for thirty minutes. The hydrolysis was stopped by adding 1N H2SO4 and plates were read using an ELISA plate reader with a 450 nm filter.

### EXAMPLE 8

### Serological Evaluation of Chickens Inoculated with Recombinant HVT

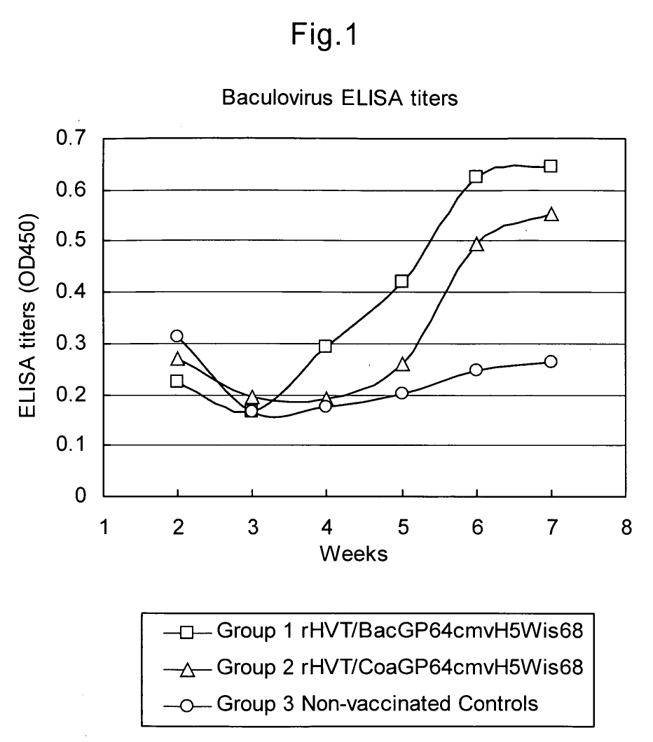

Serological responses against GP64 protein in chickens that were vaccinated with the recombinant viruses, rHVT/BacGP64CMVH5Wis68 and rHVT/CoaGP64CMVH5Wis68 were evaluated with the baculovirus ELISA system described in EXAMPLE 7. One-day-old specific pathogen free chicks (SPAFAS, Flock R-105) were vaccinated subcutaneously with one of the recombinant viruses. Group 1 was inoculated with 805 pfu per dose (0.2 ml) of rHVT/BacGP64CMVH5Wis68 (TABLE 1). Group 2 contained chickens vaccinated with 805 pfu (Group 2) of rHVT/CoaGP64CMVH5Wis68. A group of chickens (Group 3) were held as non-vaccinated negative controls. As shown in TABLE 2 and FIG. 1, ELISA titers of sera from chickens vaccinated with rHVT/BacGP64CMVH5Wis68 (Group 1) or rHVT/CoaGP64CMVH5Wis68 (Group 2) were significantly higher than those of non-vaccinated controls (Group 3) after 4 weeks old or 5 weeks old, respectively.

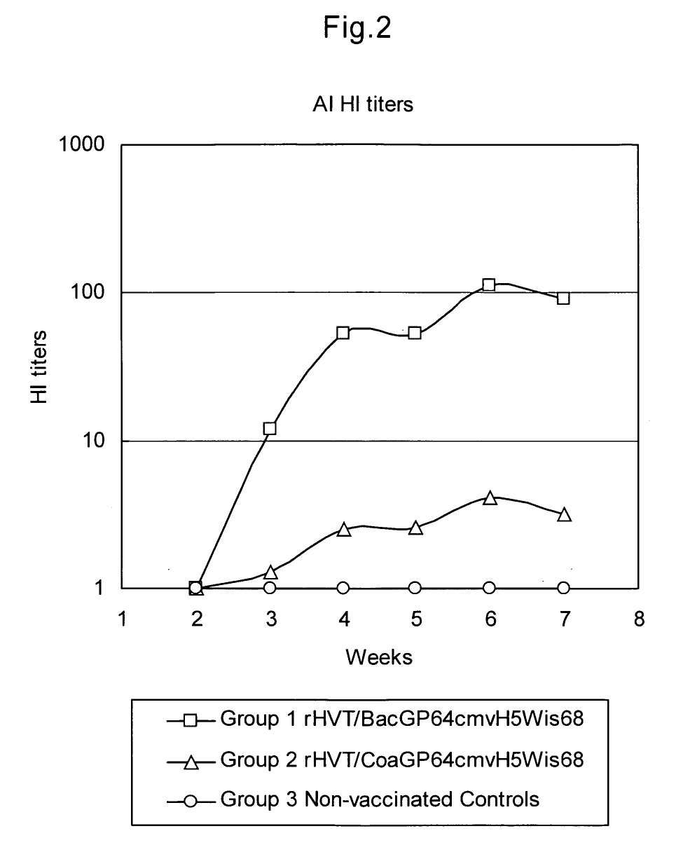

The sera from the chickens were also tested for serological responses against the other antigen gene, avian influenza virus HA gene, that was included in recombinant virus as well as the gp64 gene. The AI HI test was used for this purpose. The AI HI tests were conducted using four HA units of an inactivated A/Turkey/Wisconsin/68 (H5N9) antigen as described by D. E. Swayne et al (D. E. Swayne et al., 1998, Avian Influenza. In: A Laboratory Manual for the Isolation and Identification of Avian Pathogens, 150-155). As shown in TABLE 3 and FIG. 2, sera from chickens vaccinated with rHVT/BacGP64CMVH5Wis68 (Group 1) started to show high HI titers as early as three weeks post vaccination and the HI titers continued to increase up to the HI titer of 100 (geometric mean titer) by six weeks post vaccination. The HI titers obtained from rHVT/CoaGP64CMVH5Wis68 (Group 2) was lower than those from Group 1, although they were higher than non-vaccinated controls (Group 3).

In summary, rHVT/BacGP64CMVH5Wis68 and rHVT/CoaGP64CMVH5Wis68 were able to elicit immunity against GP64 protein, which may be able to be used to positively track vaccination, as well as immunity against the antigen protein from avian influenza virus.

TABLE 1
Treatment groups

| Group | | Age of | Vaccine | Vaccine | # of |
|---|---|---|---|---|---|
| # | Treatment Group | vaccination | dose (pfu¹) | route | chickens |
| 1 | rHVT/BacGP64CMVH5Wis68 | One-day-old | 805 | SQ² | 10 |
| 2 | rHVT/CoaGP64CMVH5Wis68 | One-day-old | 805 | SQ | 10 |
| 3 | Negative controls | One-day-old | N/A³ | SQ | 5 |

pfu¹ = plaque forming units
SQ² = subcutaneous
N/A³ = not applicable

TABLE 2
Baculovirus ELISA titers

| Group | ELISA titers (OD450) | | | | | |
|---|---|---|---|---|---|---|
| # | 2 weeks | 3 weeks | 4 weeks | 5 weeks | 6 weeks | 7 weeks |
| 1 | 0.225 | 0.166 | 0.292 | 0.421 | 0.626 | 0.645 |
| 2 | 0.270 | 0.195 | 0.192 | 0.260 | 0.496 | 0.554 |
| 3 | 0.311 | 0.167 | 0.176 | 0.201 | 0.249 | 0.263 |

TABLE 3
AI HI titers

| Group | HI titers (Geometric mean titers) | | | | | |
|---|---|---|---|---|---|---|
| # | 2 weeks | 3 weeks | 4 weeks | 5 weeks | 6 weeks | 7 weeks |
| 1 | 1 | 11.8 | 52.8 | 52.8 | 113.1 | 91.9 |
| 2 | 1 | 1.3 | 2.5 | 2.6 | 4.1 | 3.2 |
| 3 | 1 | 1 | 1 | 1 | 1 | 1 |

1. A recombinant turkey herpesvirus comprising a positive marker gene derived from an organism or virus that does not have the chicken as a host, which allows identification of vaccinated animals, inserted into a turkey herpesvirus genome.

2. A recombinant turkey herpesvirus as in claim 1, wherein said recombinant turkey herpesvirus also comprises antigen genes from avian pathogens, inserted into a turkey herpesvirus genome.

3. A recombinant turkey herpesvirus as in claim 2, wherein said positive marker gene and said avian antigen genes are inserted into a non-essential region of the turkey herpesvirus genome.

4. A recombinant turkey herpesvirus as in claim 3, wherein said non- essential region is between UL45 and UL46 of the turkey herpesvirus genome.

5. A recombinant turkey herpesvirus as in claim 1, wherein said positive marker gene is derived from Autographa calfornica nuclear polyhedrosis virus.

6. A recombinant turkey herpesyirus as in claim 1, wherein said positive marker gene is gp64 gene of Autographa californica nuclear polyhedrosis virus.

7. A recombinant turkey herpesvirus as in claim 1, wherein said positive marker gene is pp34 gene of Autographa californica nuclear polyhedrosis virus.

8. A recombinant turkey herpesvirus as in claim 2, wherein said avian antigen gene is a hemagglutinin gene of avian influenza virus.

9. A recombinant turkeyherpesvirus as in claim 8, wherein said avian influenza virus is H5 subtype.

10. A recombinant turkey herpesvirus as in claim 8, wherein said avian influenza virus is A/Turkey/Wisconsin/68 (H5N9) strain.

11. (canceled)

12. A method of identifying vaccinated animals which comprises (1) administering to chickens a recombinant turkey herpesvirus as in claim 1, (2) collectina sera from vaccinated animals, and (3) detecting humoral antibodies against a protein encoded by a positive marker gene with a serological assay.


SEQUENCE LISTING (47 sequences)

SEQ ID NO: 1 - DNA, 1539 bp, Autographa californica nuclear polyhedrosis virus
  CDS (1)..(1539): gp64 gene of Autographa californica nuclear polyhedrosis virus
atggtaagcgctattgttttatatgtgcttttggcggcggcggcgcattctgcctttgcggcggagcactgcaacgcgcaaatgaagacgggtccgtacaagattaaaaacttggacattaccccgcccaaggaaacgctgcaaaaggacgtggaaatcaccatcgtggagacggactacaacgaaaacgtgattatcggctacaaggggtactaccaggcgtatgcgtacaacggcggctcgctggatcccaacacacgcgtcgaagaaaccatgaaaacgctgaatgtgggcaaagaggatttgcttatgtggagcatcaggcagcagtgcgaggtgggcgaagagctgatcgaccgttggggcagtgacagcgacgactgttttcgcgacaacgagggccgcggccagtgggtcaaaggcaaagagttggtgaagcggcagaataacaatcactttgcgcaccacacgtgcaacaaatcgtggcgatgcggcatttccacttcgaaaatgtacagcaggctcgagtgccaggacgacacggacgagtgccaggtatacattttggacgctgagggcaaccccatcaacgtgaccgtggacactgtgcttcatcgagacggcgtgagtatgattctcaaacaaaagtctacgttcaccacgcgccaaataaaagctgcgtgtctgctcattaaagatgacaaaaataaccccgagtcggtgacacgcgaacactgtttgattgacaatgatatatatgatctttctaaaaacacgtggaactgcaagtttaacagatgcattaaacgcaaagtcgagcaccgagtcaagaagcggccgcccacttggcgccacaacgttagagccaagtacacagagggagacactgccaccaaaggcgacctgatgcatattcaagaggagctgatgtacgaaaacgatttgctgaaaatgaacattgagctgatgcatgcgcacatcaacaagctaaacaatatgctgcacgacctgatagtctccgtggccaaggtggacgagcgtttgattggcaatctcatgaacaactctgtttcttcaacatttttgtcggacgacacgtttttgctgatgccgtgcaccaatccgccggcacacaccagtaattgctacaacaacagcatctacaaagaagggcgttgggtggccaacacggactcgtcgcaatgcatagattttagcaactacaaggaactagcaattgacgacgacgtcgagttttggatcccgaccatcggcaacacgacctatcacgacagttggaaagatgccagcggctggtcgtttattgcccaacaaaaaggcaacctcataaccaccatggagaacaccaagtttggcggcgtcggcaccagtctgagcgacatcacttccatggctgaaggcgaattggccgctaaattgacttcgttcatgtttggtcatgtagttaactttgtaattatattaattgtgattttatttttgtactgtatgattagaaaccgtaatagacaatattaa

SEQ ID NO: 2 - protein, 512 aa, Autographa californica nuclear polyhedrosis virus
MVSAIVLYVLLAAAAHSAFAAEHCNAQMKTGPYKIKNLDITPPKETLQKDVEITIVETDYNENVIIGYKGYYQAYAYNGGSLDPNTRVEETMKTLNVGKEDLLMWSIRQQCEVGEELIDRWGSDSDDCFRDNEGRGQWVKGKELVKRQNNNHFAHHTCNKSWRCGISTSKMYSRLECQDDTDECQVYILDAEGNPINVTVDTVLHRDGVSMILKQKSTFTTRQIKAACLLIKDDKNNPESVTREHCLIDNDIYDLSKNTWNCKFNRCIKRKVEHRVKKRPPTWRHNVRAKYTEGDTATKGDLMHIQEELMYENDLLKMNIELMHAHINKLNNMLHDLIVSVAKVDERLIGNLMNNSVSSTFLSDDTFLLMPCTNPPAHTSNCYNNSIYKEGRWVANTDSSQCIDFSNYKELAIDDDVEFWIPTIGNTTYHDSWKDASGWSFIAQQKGNLITTMENTKFGGVGTSLSDITSMAEGELAAKLTSFMFGHVVNFVIILIVILFLYCMIRNRNRQY

SEQ ID NO: 3 - DNA, 28 bp, Artificial
  synthetic primer DNA for PCR; Primer GP64-F1
cacacaagcaagatggtaagcgctattg

SEQ ID NO: 4 - DNA, 27 bp, Artificial
  synthetic primer DNA for PCR; Primer GP64-R2
gtgggcggccgcttcttgactcggtgc

SEQ ID NO: 5 - DNA, 27 bp, Artificial
  synthetic primer DNA for PCR; Primer GP64-F2
gcaccgagtcaagaagcggccgcccac

SEQ ID NO: 6 - DNA, 26 bp, Artificial
  synthetic primer DNA for PCR; Primer GP64-R1
catttaatattgtctattacggtttc

SEQ ID NO: 7 - DNA, 17 bp, Artificial
  synthetic primer for DNA for PCR; Primer M13 Forward
gtaaaacgacggccagt

SEQ ID NO: 8 - DNA, 19 bp, Artificial
  synthetic primer DNA for PCR; Primer M13 Reverse
ggaaacagctatgaccatg

SEQ ID NO: 9 - DNA, 32 bp, Artificial
  synthetic primer DNA for PCR; Primer GP64-F3
gtcgagcaccgagtcaagaagcggccgcccac

SEQ ID NO: 10 - DNA, 22 bp, Artificial
  synthetic primer DNA for PCR; Primer GP64-R3
gatggggttgccctcagcgtcc

SEQ ID NO: 11 - DNA, 24 bp, Artificial
  synthetic primer DNA for PCR; Primer pp34-F
atatttcaaaatatgaagccgacg

SEQ ID NO: 12 - DNA, 27 bp, Artificial
  synthetic primer DNA for PCR; Primer pp34-R
gagttggtgttgttcaactcgttgcgc

SEQ ID NO: 13 - DNA, 759 bp, Autographa californica nuclear polyhedrosis virus
  CDS (1)..(759): pp34 gene of Autographa californica nuclear polyhedrosis virus
atgaagccgacgaataacgttatgttcgacgacgcgtcggtcctttggatcgacacggactacatttatcaaaatttaaaaatgcctttgcaggcgtttcaacaacttttgttcaccattccatctaaacatagaaaaatgatcaacgatgcgggcggatcgtgtcataacacggtcaaatacatggtggacatttacggagcggccgttctggttttgcgaacgccttgctcgttcgccgaccagttgttgagcacatttattgcaaacaattatttgtgctacttttaccgtcgtcgccgatcacgatcacgctcacgatcacgctcgcgatcacgttctcctcattgcagacctcgttcgcgctctcctcattgcagacctcgttcgcgatctcggtcccggtctagatcgcggtcacgttcatcgtctcccaggcgagggcgtcgacaaatattcgacgcgctggaaaagattcgtcatcaaaacgacatgttgatgagcaacgtcaaccaaataaatctcaaccaaactaatcaatttttagaattgtccaacatgatgacgggcgtgcgcaatcaaaacgtgcagctcctcgcggcgttggaaaccgctaaagatgttattttgaccagattaaacacattgcttgccgagattacagactcgttacccgacttgacgtccatgttagataaattagctgaacaattgttggacgccatcaacacggtgcagcaaacctgcgcaacgagttga

SEQ ID NO: 14 - protein, 252 aa, Autographa californica nuclear polyhedrosis virus
MKPTNNVMFDDASVLWIDTDYIYQNLKMPLQAFQQLLFTIPSKHRKMINDAGGSCHNTVKYMVDIYGAAVLVLRTPCSFADQLLSTFIANNYLCYFYRRRRSRSRSRSRSRSRSPHCRPRSRSPHCRPRSRSRSRSRSRSRSSSPRRGRRQIFDALEKIRHQNDMLMSNVNQINLNQTNQFLELSNMMTGVRNQNVQLLAALETAKDVILTRLNTLLAEITDSLPDLTSMLDKLAEQLLDAINTVQQTCATS

SEQ ID NO: 15 - DNA, 29 bp, Artificial
  synthetic primer DNA for PCR; Primer HVT45Sph
gggttatagttgcatgcgcgtggccggga

SEQ ID NO: 16 - DNA, 30 bp, Artificial
  synthetic primer DNA for PCR; Primer 45SfiR
ggccgggggggccgttagaggtgcgttttt

SEQ ID NO: 17 - DNA, 30 bp, Artificial
  synthetic primer DNA for PCR; Primer 46SfiF
cccccccggccttactgtgttttatttatc

SEQ ID NO: 18 - DNA, 28 bp, Artificial
  synthetic primer DNA for PCR; Primer HVT46Xho
catggggggctctcgagtgcagcataaa

SEQ ID NO: 19 - DNA, 52 bp, Artificial
  Oligonucleotides used for plasmid construction; Oligonucleotide Sfi-polA-Sal-Pst-Sfi-U
cggctttttatttatttaggtatgctgggggtcgacggactgcaggcccccc

SEQ ID NO: 20 - DNA, 52 bp, Artificial
  Oligonucleotides used for plasmid construction; Oligonucleotide Sfi-polA-Sal-Pst-Sfi-L
gggcctgcagtccgtcgacccccagcatacctaaataaataaaaagccgggg

SEQ ID NO: 21 - DNA, 37 bp, Artificial
  synthetic primer DNA for PCR; Primer GP64-Xba-F
cgcccttcacacaagtctagaatggtaagcgctattg

SEQ ID NO: 22 - DNA, 29 bp, Artificial
  synthetic primer DNA for PCR; Primer GP64-Sal-R
gcagaattcggtcgactattaatattgtc

SEQ ID NO: 23 - DNA, 29 bp, Artificial
  synthetic primer for DNA for PCR; Primer BamHA-F
tgacggatccatggaaagaatagtgattg

SEQ ID NO: 24 - DNA, 29 bp, Artificial
  synthetic primer for DNA for PCR; Primer SalHA-R
ctgacagtcgacctagatgcaaattctgc

SEQ ID NO: 25 - DNA, 25 bp, Artificial
  synthetic primer DNA for PCR; Primer HA-F
ctggacaatactaaggccgaacgat

SEQ ID NO: 26 - DNA, 22 bp, Artificial
  synthetic primer DNA for PCR; Primer HA-R
cactggggtctgacatttggta

SEQ ID NO: 27 - DNA, 1695 bp, Influenza A virus
  CDS (1)..(1695): Hemagglutinin gene of A/Turkey/ Wisconsin/68 (H5N9)
atggaaagaatagtgattgcccttgcaataatcagcgttgtcaaaggtgaccaaatctgcatcggttatcatgcaaacaattcaacaaaacaagttgacacaatcatggagaagaatgtgacggtcacacatgctcaagatatactggaaaaagagcacaacgggaaactctgcagtctcaaaggagtgaggcccctcattctgaaggattgcagtgtggctggatggcttcttgggaacccaatgtgtgatgagttcctaaatgtaccggaatggtcatatattgtagagaaggacaatccaaccaatggcttatgttatccgggagacttcaatgattatgaagaactgaagtatttaatgagcaacacaaaccattttgagaaaattcaaataatccctaggaactcttggtccaatcatgatgcctcatcaggagtgagctcagcatgcccatacaatggtagatcttcctttttcaggaatgtggtgtggttgatcaagaagagtaatgcatacccaacaataaagaggacctacaataacaccaatgtagaggaccttctgatattgtggggaatccatcaccctaatgatgcagcggaacaaacggaactctatcagaactcgaacacttatgtgtctgtaggaacatcaacactaaatcagaggtcaattccagaaatagctaccaggcccaaagtgaatggacaaagtggaagaatagaatttttctggacaatactaaggccgaacgatgcaatcagctttgaaagtaatgggaactttatagctcctgaatatgcatacaagatagttaaaaagggagattcagcaatcatgagaagcgaactggagtatggcaactgtgataccaaatgtcagaccccagtgggtgctataaattccagtatgccttttcacaatgttcatccccttaccattggagagtgtcccaaatatgtcaaatcagataaactggtccttgcaacaggactgaggaacgtgcctcagagagaaacaagaggtctgtttggagcaatagcaggattcatagaaggggggtggcaaggaatggtagatggatggtatggttaccatcatagcaacgagcagggaagtggatatgctgcagacaaagagtccactcagaaagcaatcgacgggatcaccaataaagtcaactcaatcattgacaaaatgaacactcaattcgaagccgttgggaaagaattcaacaacttagaaaggagaatagaaaatttgaataagaaaatggaagatggatttctagatgtatggacttacaatgcagaacttctggtgctcatggaaaatgaaagaactctggatttccatgattcatatgtcaagaacctatacgataaggtccgactccagctgagagataatgcaaaagaattgggcaatgggtgttttgagttctaccacaaatgtgacaatgaatgcatggaaagtgtgagaaacggaacgtatgactatccacaatactcagaagaatcaaggctgaacagagaggaaatagatggagtcaaattggagtcaatgggcacctatcagatactatcaatctactcaacagtggcgagttccctagcactggcaatcatggtagctggtctgtctttttggatgtgctccaatggatcattgcagtgcagaatttgcatctag

SEQ ID NO: 28 - protein, 564 aa, Influenza A virus
MERIVIALAIISVVKGDQICIGYHANNSTKQVDTIMEKNVTVTHAQDILEKEHNGKLCSLKGVRPLILKDCSVAGWLLGNPMCDEFLNVPEWSYIVEKDNPTNGLCYPGDFNDYEELKYLMSNTNHFEKIQIIPRNSWSNHDASSGVSSACPYNGRSSFFRNVVWLIKKSNAYPTIKRTYNNTNVEDLLILWGIHHPNDAAEQTELYQNSNTYVSVGTSTLNQRSIPEIATRPKVNGQSGRIEFFWTILRPNDAISFESNGNFIAPEYAYKIVKKGDSAIMRSELEYGNCDTKCQTPVGAINSSMPFHNVHPLTIGECPKYVKSDKLVLATGLRNVPQRETRGLFGAIAGFIEGGWQGMVDGWYGYHHSNEQGSGYAADKESTQKAIDGITNKVNSIIDKMNTQFEAVGKEFNNLERRIENLNKKMEDGFLDVWTYNAELLVLMENERTLDFHDSYVKNLYDKVRLQLRDNAKELGNGCFEFYHKCDNECMESVRNGTYDYPQYSEESRLNREEIDGVKLESMGTYQILSIYSTVASSLALAIMVAGLSFWMCSNGSLQCRICI

SEQ ID NO: 29 - DNA, 30 bp, Artificial
  synthetic primer DNA for PCR; Primer PrCMV1
gggctgcagagttattaatagtaatcaatt

SEQ ID NO: 30 - DNA, 25 bp, Artificial
  synthetic primer DNA for PCR; Primer PrCMV3
gggtcgttgggcggtcagccggcgg

SEQ ID NO: 31 - DNA, 25 bp, Artificial
  synthetic primer DNA for PCR; Primer PrCMV4
cttacggtaaatggcccgccggctg

SEQ ID NO: 32 - DNA, 25 bp, Artificial
  synthetic primer DNA for PCR; Primer PrCMV5
tacacttgatgtactgccaatgggc

SEQ ID NO: 33 - DNA, 25 bp, Artificial
  synthetic primer DNA for PCR; Primer PrCMV6
tatttacggtaaactgcccattggc

SEQ ID NO: 34 - DNA, 25 bp, Artificial
  synthetic primer DNA for PCR; Primer PrCMV2′
cgcgccatttaccgtcattgacgtc

SEQ ID NO: 35 - DNA, 30 bp, Artificial
  synthetic primer DNA for PCR; Primer PrCMVo1
acgtcaatgacggtaaatggcgcgcctggc

SEQ ID NO: 36 - DNA, 30 bp, Artificial
  synthetic primer DNA for PCR; Primer PrCMVR1
cgtctagaggatctgacggttcactaaacc

SEQ ID NO: 37 - DNA, 587 bp, Cytomegalovirus
  promoter (1)..(587): Cytomegalovirus immediate early promoter
agttattaatagtaatcaattacggggtcattagttcatagcccatatatggagttccgcgttacataacttacggtaaatggcccgccggctgaccgcccaacgacccccgcccattgacgtcaataatgacgtatgttcccatagtaacgccaatagggactttccattgacgtcaatgggtggagtatttacggtaaactgcccattggcagtacatcaagtgtatcatatgccaagtacgccccctattgacgtcaatgacggtaaatggcgcgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcc

SEQ ID NO: 38 - DNA, 33 bp, Artificial
  synthetic primer DNA for PCR; Primer PolyA-SalKpn
tgtggtaccgtcgacgattcacagtcccaaggc

SEQ ID NO: 39 - DNA, 33 bp, Artificial
  synthetic primer DNA for PCR; Primer PolyA-SfiF2
cttggccttattggcctaagatacattgatgag

SEQ ID NO: 40 - DNA, 39 bp, Artificial
  synthetic primer DNA for PCR; Primer PrCMVlBgl
ggtgtgttttggcccccccggctgcagagttattaatag

SEQ ID NO: 41 - DNA, 32 bp, Artificial
  synthetic primer DNA for PCR; Primer PolA-SfiR
ttggccttattggccgattcacagtcccaagg

SEQ ID NO: 42 - DNA, 22 bp, Artificial
  synthetic primer DNA for PCR; Primer HA2-P-F
ccaaatctgcatcggttatcat

SEQ ID NO: 43 - DNA, 22 bp, Artificial
  synthetic primer DNA for PCR; Primer HA2-P-R
gtttgttccgctgcatcattag

SEQ ID NO: 44 - DNA, 24 bp, Artificial
  synthetic primer DNA for PCR; Primer GP64-F
cgctgcaaaaggacgtggaaatca

SEQ ID NO: 45 - DNA, 21 bp, Artificial
  synthetic primer DNA for PCR; Primer GP64-R
caccttggccacggagactat

SEQ ID NO: 46 - DNA, 24 bp, Artificial
  synthetic primer DNA for PCR; Primer 45/46-F
tagcggcacggaaacagatagaga

SEQ ID NO: 47 - DNA, 24 bp, Artificial
  synthetic primer DNA for PCR; Primer 45/46-R
tggcgatacggttcctggtttgac